(19) United States
(12) Patent Application Publication
(10) Pub. No.: US 2022/0023187 A1
(43) Pub. Date: Jan. 27, 2022

(54) EMULSIFYING COMPOSITION CONSISTING OF AN OIL-IN-WATER EMULSIFIER AND A CYCLODEXTRIN OF SELECTED PARTICLE SIZE, CAPABLE OF PROVIDING AN OIL-IN-WATER EMULSION WITH IMPROVED SENSORY EFFECTS FOR COSMETIC USE

(71) Applicant: ROQUETTE FRERES, Lestrem (FR)

(72) Inventor: Léon MENTINK, Lille (FR)

(21) Appl. No.: 17/309,565

(22) PCT Filed: Dec. 4, 2019
(86) PCT No.: PCT/FR2019/052930

§ 371 Date: Jun. 4, 2021

(30) Foreign Application Priority Data
Dec. 5, 2018 (FR) 1872345

(51) Int. Cl. A61K 8/73; A61K 8/02; A61K 8/06

(52) U.S. Cl. CPC A61K 8/738; A61K 8/0241; A61K 2800/412; A61K 2800/10; A61K 8/062

(57) ABSTRACT

The present invention relates to a sensory emulsifying composition, in particular for cosmetic use, capable of making it possible to obtain an oil-in-water-type emulsion, comprising at least one cyclodextrin in the form of solid particles, said particles having a volume-average size d(4,3) measured by laser granulometry, of between 2 and 20 μm, preferably between 3 and 12 μm, and more preferably between 4 and 8 μm; and at least one emulsifier of natural origin selected from oil-in-water emulsifiers, having an HLB greater than or equal to 8, and most preferably greater than or equal to 9.

The present invention relates to an emulsifying composition of plant origin, which is ready to use and can be used directly when cold, with particular uses in the field of cosmetics. This composition comprises at least one cyclodextrin in the form of solid particles, said particles having a volume-average size d(4.3) measured by laser granulometry, less than or equal to 20 μm, and at least one emulsifier of natural origin. The emulsifying composition according to the invention can be used as a cosmetic composition as such, or as a premix for producing and stabilizing an emulsion. This emulsifying composition enables in particular the easy production of very fine liquid oil-in-water (O/W) emulsions, having varied textures, that are compatible with the skin. The compositions obtained have a soft, smooth feel, even when they have a high content of fatty phase in the emulsion, and spread easily, without pilling.

An emulsion is a dispersion of a liquid (or a material rendered liquid) in fine droplets in another liquid that is immiscible with the former. It has a macroscopically homogeneous appearance, but appears heterogeneous under the microscope. The liquid in the form of droplets is referred to as dispersed (or discontinuous) phase, while the other liquid is referred to as dispersing (or continuous) phase. In general terms, an emulsion consists of two phases (simple emulsion): a hydrophilic (aqueous) phase and a lipophilic (oil) phase. An oil-in-water emulsion is referred to as a direct emulsion.

Emulsions are broadly used in the field of cosmetics. As a cosmetic product, these emulsions must meet the needs of consumers, who require effectiveness and safety as well as pleasant sensory properties. In order to meet these demands, the formulations of emulsions have increased in complexity over time, integrating ever more functional or sensory ingredients of synthetic origin, generally derived from petrochemicals.

It is known that most cosmetic compositions in emulsion form, typically simple emulsions, are stabilized by surfactants. By way of example, document EP 0 685 227 proposes a highly complex system of sunscreen cosmetic compositions, comprising an aqueous continuous phase, a protective system capable of filtering UV rays, a surfactant, organic solvents (lower polyols and alcohols) and at least one polymer or more particularly a cross-linked copolymer (alkyl acrylates, vinyl acetate). Document FR 2 858 777 in turn describes an oil-in-water emulsion containing at least one fatty product (fatty acid esters, waxes, butters, natural oils—vegetable, animal, of marine, synthetic or mineral origin, hydrogenated oils and the mixtures thereof), at least one surfactant (ethoxylated polyglycerol fatty acid esters, alcohol ethoxylates), at least one co-surfactant and water.

However, the use of surfactants in products intended for use with humans or animals, whether topically or orally, may be problematic. Indeed, surfactants may damage cell membranes. Thus, efforts are being made, in particular in the field of cosmetics, to reduce the potentially harmful effects of surfactants.

Moreover, cosmetic products currently need to address a new expectation among consumers: the natural origin and, even more demandingly, the naturalness of the compositions. Indeed, consumers are now seeking cosmetic products consisting essentially of ingredients that are natural or of natural origin, having minimal chemical modifications or synthetic or petrochemical grafts.

Also, one of the aims of the present invention is to provide a composition that allows the formation of stable emulsions, even according to a “cold” process, by dispersion in an aqueous phase and subsequent addition of an oil or fatty product. Such a composition makes it possible to avoid the use of non-biodegradable petroleum-based surfactants, in particular glycol derivatives and ethoxylated derivatives. The composition according to the present invention also makes it possible to manufacture Pickering-type emulsions. Emulsions of this type lack a surfactant and are stabilized by colloidal microparticles, generally silicas, which are placed at the interfaces of the continuous phase and the dispersed phase. In the context of the present invention, these colloidal particles appear to be organic particles consisting of inclusion complexes between at least one cyclodextrin and at least one fat molecule. These particles are very advantageously compatible with the skin and the hair and do not damage cell membranes.

This result is all the more remarkable since the prior art shows that the production of emulsifying compositions for cosmetic use containing cyclodextrins does not make it possible to obtain emulsions easily and directly when cold; until now, it was necessary to use petroleum-based surfactants. This is, in particular, what is mentioned in document EP 2 091 502 B1 which describes an oil-in-water (O/W) emulsion containing water, a fatty substance, a modified polysaccharide, and a cyclodextrin, the essential feature of this O/W emulsion being that it contains surfactants with a molecular weight of less than 5000 g/mol and in an amount of less than 2% by weight. It is neither known nor obvious to produce emulsifying systems containing cyclodextrins that make it possible to obtain very fine and very stable emulsions, and to do so without using petroleum-based surfactants.

Furthermore, the main appeal of the cosmetic product for the consumer lies in its sensory properties, long before the beneficial effects can be observed. The challenge that a cosmetic product needs to meet is therefore that of affording a beneficial cosmetic effect while providing, before or during the application, the most pleasant sensations possible. However, the removal of functional or sensory ingredients deriving from petrochemicals, their substitution by ingredients of natural origin, or the introduction of new ingredients that are natural or of natural origin, may entail a degradation of the sensory properties of the cosmetic product to a greater or lesser extent, in particular with regard to their appearance, the way in which the product is absorbed, their application or their properties once they have been applied to the skin or the hair and nails. Thus, compositions that meet the criteria of naturalness may be difficult to spread, tend to pill, or feel squeaky, brittle, or even insufficiently slippery when applied. These insufficient or degraded sensory perceptions are detrimental to the quality or image of a cosmetic product.

A previous application filed by the applicant, FR1853362 as yet unpublished, relates to an emulsifying composition comprising a cyclodextrin and an emulsifier of natural origin having a HLB greater than or equal to 8. The beta-cyclodextrin used is a beta-cyclodextrin marketed by Roquette Freres under the name “Beauté by Roquette® CD 102”, with a volume-average size equal to 95 μm. However, the sensory properties obtained by means of the composition implemented in this application are not entirely satisfactory.

Thus, one objective of the present invention is to provide an improved emulsifying composition, for cosmetic use, offering improved sensory properties compared with the emulsifying composition of application FR1853362, in particular as regards the smoothness effect, the ease of spreading, the softness of touch, the squeakiness on application, the penetration of the composition and the pilling.

Another aim of the present invention is to provide a composition of 100% natural origin. The natural origin of the ingredients used to formulate products for everyday use such as cosmetic compositions is currently a major issue, not only as regards safeguarding and protecting our environment, but also for the wellbeing of the consumers. In this regard, the composition according to the present invention makes it possible to replace synthetic and in particular petroleum-based surfactants, in particular ethoxylated surfactants, which are nowadays sought to be replaced for environmental reasons, due to their poor biodegradability, and for safety reasons, due to the dangerous nature of ethylene oxide which is widely used to produce polyethoxylated surfactants, which is toxic and flammable).

Another objective of the present invention is to provide a composition that is ready to use, allowing very straightforward implementation by the formulator, with minimal energy input, in particular by inserting all the ingredients into a single tank or reactor (so-called “one-pot” formulation). As regards its implementation, the emulsifying composition that is the object of the present invention can advantageously be used according to a “cold process”, that is even at room temperature. “Cold process” is understood to mean that the emulsifying composition can be implemented directly by dispersion in water at a water temperature of less than 45° C., preferably of less than 35° C. and better still at room temperature.

Another objective of the present invention is to provide a composition for broad spectrum cosmetic use, that is versatile from the point of view of the end products considered: from this point of view, the composition according to the invention can be used in a variety of products including lotions, creams, gels, milks, etc. Furthermore, said composition is advantageously non-irritating and non-allergenic for the skin. It also has the advantage of not being dependent on the pH or the presence of electrolytes: in other words, its emulsifying capacity is not affected by the pH of the environment, nor by the presence of mono-, di- or trivalent salts. This criterion is all the more important since, in general, products for cosmetic use and in particular for topical application are likely to be subjected or exposed to pH variations (for example, the pH of the skin is slightly acidic, and varies between 4 and 6). Having an emulsifying composition that has no particular limit on use in terms of pH is therefore a major technical advantage for a cosmetic composition.

All of these objectives, which constitute a complex technical problem to be solved, are finally achieved through the main object of the present invention, which consists of an emulsifying composition, in particular for cosmetic use, capable of making it possible to obtain a liquid oil-in-water-type (O/W) emulsion, comprising:

a) at least one cyclodextrin in the form of solid particles, said particles having a volume-average size d(4.3) measured by laser granulometry, of between 2 and 20 μm, preferably of between 3 and 12 μm, and more preferably of between 4 and 8 μm,
b) and at least one emulsifier of natural origin, selected from oil-in-water-type (O/W) emulsifiers, having a hydrophilic-lipophilic balance greater than or equal to 8, preferably greater than or equal to 9.

The calculation of the HLB takes into account the molecular masses of the hydrophilic parts and the molecular masses of the molecule under consideration and can be obtained according to the following equation:

$$HLB = 20 \frac{Molecular mass of the hydrophilic part}{Molecular mass of the molecule} \qquad [ Math . 1 ]$$

Cyclodextrin

The composition according to the invention implements at least one cyclodextrin in the form of solid particles, said particles having a volume average size d(4.3) measured by laser granulometry, of less than or equal to 20 μm, preferably of less than or equal to 12 μm, and most preferably of less than or equal to 8 μm.

In the present application, the term “cylcodextrin” designates and includes any one of the cyclodextrins known to a skilled person, such as the native and unsubstituted cyclodextrins containing 6 to 12 glucose units linked by covalent bonds between carbons 1 and 4, and in particular the alpha-, beta- and gamma-cyclodextrins respectively containing 6, 7 and 8 glucose units.

This term also covers “cyclodextrin derivatives”, namely molecules of which a part at least of the OH-hydroxyl groups has been transformed into OR groups, where R generally designates an alkyl group. From this point of view, the cyclodextrin derivatives include in particular the methylated and ethylated cyclodextrins, but also those substituted with a hydroxyalkyl group such as hydroxypropylated and hydroxyethylated cyclodextrins.

The preferred cyclodextrins according to the present invention are the alpha-, beta- and gamma-cyclodextrins. According to a preferred embodiment, the cyclodextrin used in the composition according to the invention is a beta-cyclodextrin, preferably “native”, that is the hydroxyl groups of which are not chemically substituted.

The cyclodextrin may in particular be provided in the form of a crystalline, pseudo-crystalline or amorphous powder.

In the context of the present invention, the cyclodextrin is present in the form of solid particles characterized by a volume average size d(4.3), also referred to as volume-average diameter, measured by laser granulometry, of less than or equal to 20 μm, preferably of less than or equal to 12 μm, and most preferably of less than or equal to 8 μm. According to a preferred embodiment, the volume-average size of the solid particles of cyclodextrin is of between 2 μm and 20 μm, preferably of between 3 μm and 12 μm, and most preferably of between 4 μm and 8 μm.

The solid cyclodextrin particles can have any geometric shape, regular or irregular, and can be well individualized cyclodextrin crystals or agglomerates of cyclodextrin crystals linked together by crystal bridges. Preferably, the solid cyclodextrin particles have regular geometric shapes.

The volume-average size, generally denoted denoted d(4.3), is calculated according to standard ISO 9276-2:2014 based on the volumetric particle size distributions, measured by laser diffraction granulometry, for example using a laser particle size analyzer of the MasterSizer® range, for example “Mastersizer 2000™”, “Mastersizer 3000™”, “Mastersizer 3000E™” from the company Malvern Instruments®, or a laser particle size analyzer “Particula LA960” from the company Horiba©. These methods of measuring by laser diffraction can be implemented in a wet process or a dry process, according to the guidelines of standard ISO 13320:2009. When the wet process is used, it is recommended to use 2-propanol as measurement fluid.

Preferably, the solid cyclodextrin particles have a volumetric size distribution, measured by laser diffraction granulometry, in which the characteristic diameters d(10), d(50) and d(90) are such that:

a) the diameter d(10) of between 0.8 and 5.0 μm, preferably of between 1.0 and 2.5 μm, and
b) the diameter d(50) is between 5 and 15.0 μm, preferably of between 7 and 10.0 μm, and
c) the diameter d(90) is between 15 and 30.0 μm, preferably of between 20 and 25.0 μm.

The characteristic diameters d(10), d(50) and d(90), as defined in standard ISO 13320:2009 under the notations ×10, ×50 and ×90, are the particle diameters corresponding respectively to 10%, 50% and 90% of the volumetric cumulative size distribution.

According to a preferred embodiment, the solid cyclodextrin particles have a volume-size distribution, measured by laser diffraction granulometry, the characteristic diameters d(10), d(50) and d(90) of which are such that:

a) the diameter d(10) is of less than or equal to 5.0 μm, preferably of less than or equal to 2.5 μm,
b) And the diameter d(50) is of less than or equal to 15.0 μm, preferably of less than or equal to 10.0 μm,
c) And the diameter d(90) is of less than or equal to 30.0 μm, preferably of less than or equal to 25.0 μm.

Even more preferably, the solid cyclodextrin particles have a volume-size distribution, measured by laser diffraction granulometry, with a coefficient of variation of less than or equal to 100%, preferably of less than or equal to 90%, and most preferably of less than or equal to 73%. As defined in standard ISO 13320:2009, the coefficient of variation of the particle size distribution is the standard deviation of the particle size distribution divided by the volume-average size d(4.3), also referred to as volume-average diameter.

According to this preferred embodiment, the cyclodextrin is thus provided in the form of solid particles the volume-size distribution of which measured by laser diffraction granulometry, has the following characteristics:

a) A volume-average size d(4.3) of between 2 and 20 μm, preferably of between 3 and 12 μm, and more preferably of between 4 and 8 μm,
b) A coefficient of variation less than or equal to 100%, preferably less than or equal to 90%, and more preferably less than or equal to 73%.

Thanks to this specific size selected, the cyclodextrin particles contribute to improving the sensory perception of touch, particularly by increasing the sensation of softness. Moreover, this use allows a clear reduction of the feeling of squeakiness upon application, as well as a reduction of pilling, even a disappearance of this pilling depending on the nature of the skins.

Oil-in-Water Emulsifier of Natural Origin

In the present application, the term oil-in-water emulsifier “of natural origin” designates any emulsifier obtained from renewable resources, in particular extracted from or secreted by plants, micro-organisms or algae, and capable of making it possible, after physical, chemical or enzymatic modification, to obtain an oil-in-water-type emulsion.

The oil-in-water emulsifier of natural origin having a hydrophilic-lipophilic balance (HLB) greater than or equal to 8, preferably greater than or equal to 9, is preferably provided in the emulsifying composition in an oil-in-water emulsifier/cyclodextrin ratio (weight/weight) of between 0.01:1 and 1:1, preferably of between 0.05:1 and 0.5:1, more preferably of between 0.10:1 and 0.35:1 and better still of between 0.15:1 and 0.30:1.

This O/W emulsifier of natural origin preferably has a hydrophilic-lipophilic balance (HLB) of between 8 and 20, preferably of between 9 and 16, and better still of between 11 and 14. It can also be selected from products that are naturally biodegradable in a hydrated natural environment.

In particular, the average HLB of the whole emulsifying system of said composition is greater than or equal to 8, preferably greater than or equal to 9. The average HLB of the whole emulsifying system of the composition is calculated by taking a weighted average of the HLB of each emulsifier, weighting the HLB of each emulsifier present by its mass fraction with respect to the total weight of emulsifiers present.

This O/W emulsifier of natural origin is preferably selected from the following products, as long as they meet the HLB condition above: the alkyl polyglucosides; the mixtures of at least one alkyl polyglucoside and at least one fatty alcohol; the non-ethoxylated polyol fatty esters, and in particular from the glycerol, polyglycerol, sorbitol, sorbitan, anhydrohexitol such as in particular isosorbide, mannitol, xylitol, erythritol, maltitol, sucrose, glucose, polydextrose non-ethoxylated fatty esters, non-ethoxylated fatty esters of hydrogenated glucose syrups, dextrin non-ethoxylated fatty esters and non-ethoxylated fatty esters of hydrolyzed starches.

The O/W emulsifier of natural origin is preferably selected to be naturally biodegradable in a hydrated natural environment. In particular, it can be non-ethoxylated polyol fatty esters obtained from fatty acids or by transesterification from an oil or oil mixtures. The fatty acids used comprise 8 to 22 carbon atoms, preferably 10 to 18 carbon atoms, and in particular 12 to 18 carbon atoms. These acids can be linear or branched, saturated or unsaturated, having one or more lateral hydroxyl functions. The oils can be saturated or unsaturated, from liquid to solid at room temperature, and optionally have hydroxyl functions, preferably with an iodine index between 1 and 145, and in particular of 5 to 105.

The O/W emulsifier of natural origin can be in particular selected from the polyglycerol esters, and preferably from the esters resulting from the reaction of polyglycerols comprising 2 to 12 glycerol units, preferably 3 to 10 glycerol units with at least one partially hydrogenated or non-hydrogenated vegetable oil with an iodine index between 1 and 15, and in particular of 5 to 10. It can be, in particular, oleic, stearic, palmitic, lauric, diisostearic and caprylic esters of polyglycerols and in particular the following products: Polyglyceryl-5 Dioleate with a HLB around 8 (like Dermofeel® G 5 DO from Evonik Dr. Straetmans GmbH), Polyglyceryl-2 Caprate preferably having a HLB of around 9 (like HYDRIOL® PGC.2 from HYDRIOR), Polyglyceryl-3 Stearate preferably having a HLB of around 9 (like Dermofeel® PS from Evonik Dr. Straetmans GmbH), Polyglyceryl-2 Laurate preferably having a HLB of around 9 (like Dermofeel® G2L from Evonik Dr. Straetmans GmbH), Polyglyceryl-3 Palmitate preferably having a HLB of around 10 (like Dermofeel® PP from Evonik Dr. Straetmans GmbH), Polyglyceryl-10 Diisostearate preferably having a HLB of around 11 (like Dermofeel® G10 DI from Evonik Dr. Straetmans GmbH), Polyglyceryl-6 Caprylate preferably having a HLB of around 11.5, Polyglyceryl-5 Laurate preferably having a HLB of around 13 (like Dermofeel® G5L from Evonik Dr. Straetmans GmbH), Polyglyceryl-3 Caprate preferably having a HLB of around 14 (like HYDRIOL® PGC.3 from HYDRIOR), Polyglyceryl-4 Caprate preferably having a HLB of around 14 (like MASSOCARE PG4 C from Masso), Polyglyceryl-10 Monolaurate preferably having a HLB of around 14.8, Polyglyceryl-6 Caprylate preferably having a HLB of around 15 (like Dermofeel® G 6 CY from Dr. Straetmans GmbH/Evonik), Polyglyceryl-10 Laurate preferably having a HLB of around 16 (like Dermofeel® G 10 L from Dr. Straetmans GmbH/Evonik).

The O/W emulsifier of natural origin is preferably selected from the alkyl polyglucosides, sometimes also referred to as alkyl polyglycosides, and denoted by the acronym APG. These emulsifiers are non-ionic surfactants that are well known per se. Patent FR 2 948 285 presents them in terms of structure, and explains how to prepare them. They can be represented by the following general formula (I): R1-O—(R2-O)p-(S)n, wherein:
S is a reducing saccharide, which can comprise 5 to 6 carbon atoms,
R1 denotes a linear or branched alkyl and/or alkenyl radical, preferably having around 8 to 24 carbon atoms, or an alkyl phenyl radical, preferably an alkyl phenyl radical in which the linear or branched alkyl group includes around 8 to 24 carbon atoms,
R2 denotes an alkylene radical having 2 to 4 carbon atoms,
n denotes a value ranging from 1 to 15,
p denotes a value ranging from 0 to 10.

Reducing saccharide relates, in formula (I), to the saccharide derivatives which do not have in their structures a glycosidic bond established between an anomeric carbon and the oxygen of an acetal group as defined in the reference work: “Biochemistry”, Daniel Voet/Judith G. Voet, p. 250, John Wyley & Sons, 1990. The oligomeric structure (S)n, can be provided as any form of isomer, be it an optical, geometric or positional isomer; it can also represent a mixture of isomers.

According to one particular aspect of the present invention, in the definition of the compounds of formulas (I), S represents a reducing saccharide selected from glucose, dextrose, sucrose, fructose, idose, gulose, galactose, maltose, isomaltose, maltotriose, lactose, cellobiose, mannose, ribose, xylose, arabinose, lyxose, allose, altrose, dextrane or tallose and more particularly a reducing saccharide selected from glucose, xylose or arabinose.

A first preferred alternative of alkyl polyglucosides according to the present invention are the C12-C20 alkyl glucosides, that is the compounds of formula (I) wherein:
R1 denotes more specifically a linear or branched alkyl and/or alkenyl radical having around 12 to 20 carbon atoms
R2 denotes an alkylene radical having 2 to 4 carbon atoms,
p takes a value ranging from 0 to 3, preferably equal to zero,
S denotes glucose, fructose or galactose, more preferably glucose.

A second preferred alternative of the alkyl polyglucosides according to the present invention are the C12-C20 alkyl glucosides of the first preferred alternative wherein:
R1 denotes more particularly a linear alkyl radical including about 12 to 20 carbon atoms
p is equal to zero,
S denotes glucose

Alkyl polyglucosides of formula (I) are in particular commercially available with the following names: Plantacare® 810 UP (R1 is C8-C10/INCI: caprylyl/capryl glucoside), Plantacare® 818 UP (R1 is C8-C16/INCI: Coco-glucoside), Plantacare® 2000 UP (R1 is C8-C16/INCI: decyl glucoside) and Plantacare® 1200 UP (R1 is C12-C16/INCI: lauryl glucoside) sold by the company BASF; Macanol® 810 (R1 is C8-C10), Macanol® 1200 (R1 is C12-C14), Macanol® 816 (mixture of R1 is C8, C10, C12, C14, C16) sold by the company FCI Technology; Neocare MF 0718 (R1 is C8-C10/INCI: caprylyl/capryl glucoside), Neocare MF 0012 (R1 is C12-C14/INCI: lauryl glucoside), Neocare MF 0002 (R1 is C8-C16/INCI: decyl glucoside), Neocare MF 818 (R1 is C8-C16/INCI: coco glucoside) sold by the company Neochem; Tego Care CG 90 (R1 is C14-C16/INCI: cetearyl glucoside) sold by the company Evonik Healthcare.

The O/W emulsifier of natural origin preferably is a mixture consisting of at least one alkyl polyglucoside and at least one fatty alcohol. In these mixtures, the alkyl polyglucosides can be selected from all the alkyl polyglucosides useful to the invention described beforehand. The fatty alcohols useful for mixing with the alkyl polyglucosides include the straight or branched fatty alcohols having a total number of carbon atoms ranging from 8 to 24.

Mixtures of alkyl polyglucosides and fatty alcohols useful for the invention and commercially available are those sold by the company SEPPIC: Montanov™ 14 (INCI: Myristyl Alcohol & Myristyl Glucoside), Montanov™ 202 (INCI: Arachidyl Alcohol and Behenyl Alcohol and Arachidyl Glucoside), Montanov™ 68 (INCI: Cetearyl Alcohol & Cetearyl Glucoside), Montanov™ 82 (INCI: Cetearyl Alcohol and Coco-Glucoside), Montanov™ S (INCI: Coco-Glucoside & Coconut Alcohol), Montanov™ L (INCI: C14-22 Alcohols & C12-Alkyl Glucoside).

The preferred mixture of alkyl polyglucoside and fatty alcohol is that sold by SEPPIC under the name “Montanov™ L”, which is a mixture of C14-C22 fatty alcohols and C12-C20 alkyl polyglucosides (INCI: C14-22 Alcohols & C12-20 Alkyl Glucoside).

A preferred alternative of the emulsifying composition according to the invention also comprises as O/W emulsifier of natural origin a mixture consisting of at least one alkyl polyglucoside and at least one fatty alcohol, preferably at least one C12-C20 alkyl polyglucoside and at least one linear or branched fatty alcohol having a total number of carbon atoms ranging from 8 to 24, and most preferably a mixture of C14-C22 fatty alcohol and C12-C20 alkyl glucoside.

The O/W emulsifier of natural origin can likewise be selected from dextrin or hydrolyzed starch fatty esters, in particular in the form of sodium octenylsuccinate esters. It may be, for example, products sold by the Applicant under the names CLEARGUM® and in particular the products CLEARGUM® CO 01 and CLEARGUM® CO 03.

Effects of Selecting the Particle Size of Cyclodextrin

Compared with an emulsifying composition comprising a cyclodextrin with a volume-average size greater than or equal to 90 μm, such as the beta-cyclodextrin “Beauté by Roquette® CD 102” marketed by Roquette Frères, the emulsifying composition according to the invention makes it possible to obtain emulsions that are easier to spread, having a smoother, softer feel, a much lower squeakiness, faster penetration into the keratinous materials, and leading to little or no pilling. Optionally, the emulsifying composition according to the invention also makes it possible to obtain interesting sensory effects, such as a particular texture or a feeling of freshness, according to the proportions used.

The interesting properties of said emulsifying composition are the result of the combination of the two compounds used which have a good synergy, both in terms of emulsion stability and sensory properties. Although very satisfactory results are obtained regardless of the proportions in which these compounds are combined, the results are especially convincing when they are combined in a very precise ratio.

In particular, each of the different components of the emulsifying composition according to the invention can be integrated into a different phase of the final emulsion, before proceeding to the emulsification. Alternatively, the various compounds of the present composition according to the invention are mixed together to constitute a premix, said premix being able to be added to any of the phases of the emulsion in which the premix will be used. The invention advantageously allows these two embodiments with the same compounds, which allows for increased flexibility and ease of use.

The emulsifying composition according to the invention has in particular the advantage of being completely of natural origin, and capable of being used in a “cold” process (i.e. used at room temperature). Said composition according to the invention is, in particular, for cosmetic use and, in this regard, is not sensitive to minor variations of pH or salinity in the environment, it is not irritating and is not likely to cause allergies, especially to the skin. Moreover, the composition according to the invention can be used to produce any type of emulsion, in particular Pickering-type emulsions, and thus is suited to a broad range of uses: creams, milks, serums, lotions, etc.

Water

The emulsifying composition can contain water in a so-called “combined or bound” form and/or in a so-called “free” form. Combined or bound water consists of water molecules included in the crystalline structure of the cyclodextrin and/or polyol powders, and of water molecules adsorbed on the surface of these powders by physical hydration equilibrium. Free water consists of water molecules that can circulate freely between the cyclodextrin and/or polyol powders. This free water can in particular place the cyclodextrin and/or polyol powders in suspension.

According to one embodiment, the emulsifying composition comprises a combined or bound water content between 1% and 25%, by weight relative to the total weight of said emulsifying composition. Preferably, the combined or bound water content is between 2% and 15%, most preferably of between 3% and 10%.

According to one embodiment, the emulsifying composition can comprise a free water content of less than or equal to 50% by weight relative to the total weight of said emulsifying composition. Preferably, this free water content is of less than or equal to 40%, more preferably to 30%, and most preferably to 20%.

Polyol

According to one embodiment, the emulsifying composition likewise comprises at least one polyol.

The polyols referred to in the present Application are all the polyols known, and in particular maltitol, mannitol, xylitol, erythritol, sorbitol, glycerol, glycerol and sorbitol being the preferred polyols. Preferably, this polyol is crystallized or else is in the form of a powder.

Thus, in particular, the invention relates to an emulsifying composition, in particular for cosmetic use and capable of making it possible to obtain a liquid oil-in-water-type (O/W) emulsion which comprises, or preferably consists of:

1) 40% to 95% by weight, relative to the total weight of the composition, of at least one cyclodextrin in the form of solid particles, said particles having a volume-average size d(4.3) measured by laser granulometry, of between 2 and 20 μm, preferably of between 3 and 12 μm, and more preferably of between 4 and 8 μm,
2) 5% to 40% by weight, relative to the total weight of the composition of at least one emulsifier of natural origin selected from oil-in-water emulsifiers, having a HLB greater than or equal to 8, and more preferably an alkyl polyglucoside having a HLB greater than or equal to 9,
3) and 0% to 40% by weight, relative to the total weight of the composition of at least one polyol.

Preferably, this emulsifying composition according to the present invention comprises, or preferably consists of:

1) 45% to 85% by weight, relative to the total weight of the composition of at least one cyclodextrin in the form of solid particles, said particles having a volume-average size average particle size d(4.3) measured by laser granulometry, of between 2 and 20 μm, preferably of between 3 and 12 μm, and more preferably of between 4 and 8 μm,
2) 5% to 30% by weight, relative to the total weight of the composition of at least one emulsifier of natural origin selected from oil-in-water emulsifiers, having a HLB greater than or equal to 8, and more preferably an alkyl polyglucoside having a HLB greater than or equal to 9,
3) and 10% to 40% by weight, relative to the total weight of the composition of at least one polyol.

Most preferably, this composition according to the present invention comprises, or preferably consists of:

1) 40% to 80% by weight, relative to the total weight of the composition of at least one cyclodextrin in the form of solid particles, said particles having a volume-average size d(4.3) measured by laser granulometry, of between 2 and 20 μm, preferably of between 3 and 12 μm, and more preferably of between 4 and 8 μm,
2) 10% to 20% by weight, relative to the total weight of the composition of at least one emulsifier of natural origin selected from oil-in-water emulsifiers, having a HLB greater than or equal to 8, and more preferably an alkyl polyglucoside having a HLB greater than or equal to 9,
3) and 10% to 30% by weight, relative to the total weight of the composition of at least one polyol.

In the preceding three preferred embodiments of the emulsifying composition, it is preferred for the oil-in-water emulsifier of natural origin to be selected from: at least one alkyl polyglucoside; or at least one mixture of at least one alkyl polyglucoside and at least one fatty alcohol; or at least one non-ethoxylated polyol fatty esters. more preferably, the emulsifier is selected from the mixtures of at least one alkyl polyglucoside and at least one fatty alcohol.

Emulsion, Preferably a Pickering-Type Emulsion, Implementing the Emulsifying Composition

The emulsifying composition according to the invention makes it possible to produce emulsions, preferably Pickering-type emulsions, which can advantageously be stabilized by organic particles that are compatible with the skin or the hair. So-called “Pickering” emulsions are obtained by replacing the surfactants with emulsifying systems made up of solid microparticles combined with fatty products.

Without wishing to be bound to any one theory, it appears that the presence of very small amounts of an oil-in-water emulsifier of natural origin, greatly facilitates the in situ formation of inclusion complexes between cyclodextrin and certain specific molecules present in the dispersed fatty phase, and this in the form of colloidal or solid particles, which are placed at the oil and water interfaces. These particles are very physically and sensorially compatible with the skin or the hair and do not damage cell membranes.

The combination of cyclodextrin with small amounts of an oil-in-water emulsifier of natural origin having a hydrophilic-lipophilic balance greater than or equal to 8, preferably greater than or equal to 9, makes it possible to obtain highly stable emulsions with droplets having a size of less than 30 μm, or of less than 10 μm.

The emulsifying composition according to the invention can additionally comprise other products capable of forming or stabilizing Pickering emulsions such as silicas and octenylsuccinate starches in the form of calcium or aluminum salts.

Thus, another object of the present invention relates to an emulsion, preferably an oil-in-water (O/W) Pickering-type emulsion, in particular for cosmetic use, characterized in that it contains at least one cyclodextrin in the form of solid particles, said particles having a volume-average size d(4.3) measured by laser granulometry, of less than or equal to 20 μm, more preferably of between 2 and 20 μm, preferably of between 3 and 12 μm, and more preferably of between 4 and 8 μm,

and at least one emulsifier of natural origin having a HLB greater than or equal to 8, preferably greater than or equal to 9, in an emulsifier/cyclodextrin ratio (weight/weight) of between 0.01:1 and 1:1, preferably of between 0.15:1 and 0.30:1. Said emulsifier of natural origin is preferably selected from the alkyl polyglucosides, the mixtures of at least one alkyl polyglucoside and at least one fatty alcohol, and the non-ethoxylated polyol fatty esters,

The emulsion, in particular a Pickering emulsion, using the emulsifying composition according to the invention, comprises an aqueous phase which, further, consists of water.

According to one embodiment qualified as “water rich”, the water content in the emulsion is between 50% and 95%, preferably of between 60% and 92%, most preferably of between 65% and 90%, by weight relative to the total weight of the emulsion. The emulsion according to this embodiment is ready to use, and thus can be used without the user adding additional water.

According to one embodiment qualified as “water poor”, the water content in the emulsion is between 2% and 50%, preferably of between 5% and 35%, most preferably of between 10% and 40%, by weight relative to the total weight of the emulsion. The emulsion according to this embodiment has the advantage of reducing the total mass of containers such as flasks, bottles or jars, by reducing the water mass in the emulsion. This thus reduces transport costs and the amount of exhaust gases released by such transport. The user may need to add water at the time of use to ensure correct use of this embodiment of the emulsion.

The emulsion, in particular a Pickering O/W emulsion, implementing the emulsifying composition according to the invention can also comprise a fatty phase that can be liquid at room temperature (25° C.), for example such as vegetable oils, or solid as in the case of waxes. This liquid fatty phase can be of mineral, animal, vegetable or synthetic origin and consist of hydrocarbon oils or optionally silicone oils. Hydrocarbon oil is understood to mean an oil formed essentially, or consisting of, carbon and hydrogen atoms and optionally oxygen and nitrogen atoms, which can contain alcohol, ester, ether, carboxylic acid, amine and/or amide groups.

Preferably, the emulsion implementing the emulsifying composition according to the invention can comprise one or more oils, preferably at least one non-volatile liquid oil. Non-volatile liquid oil is understood to mean an oil susceptible to remain on the skin at room temperature, at atmospheric pressure for at least one hour.

The liquid fatty phase advantageously comprises one or more non-volatile oils which provide an emollient effect on the skin. These can include fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyl decyl palmitate, 2-octyldodecyl myristate or lactate, 2-diethyl hexyl succinate, diisostearyl malate, tracetin, tricaprin, caprylic/capric acid triglycerides, glycerin triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, “mals” oil, wheat germ oil, rapeseed oil, sunflower oil, cottonseed oil, jojoba oil, peanut oil, olive oil and mixtures thereof, vegetable butters such as shea butter and camellia butter.

These oils can be hydrocarbonized or siliconized type oils such as kerosene oil, squalane oil, petrolatum, dimethyl siloxanes and mixtures thereof.

The liquid fatty phase can also optionally comprise volatile oils. Volatile oil is understood to mean an oil that can evaporate from the skin, in less than one hour at room temperature and atmospheric pressure. The volatile oils can be, for example, selected from silicone oils or short fatty acid triglycerides in order to reduce the greasy feel.

Preferably the emulsion-type composition, in particular an O/W Pickering emulsion, implementing the emulsifying composition according to the invention only contains oils of renewable origin and in particular oils or butters of plant origin, preferably refined. These oils and butters are perfectly suited to the emulsifying system used in the emulsifying composition of the invention in the sense that they make it possible to obtain very stable emulsions with high whiteness and easily adjustable viscosity. The emulsifying composition according to the invention advantageously makes it possible to prepare oil-in-water emulsions with very high oil content. This type of oil-rich O/W emulsions is normally difficult to obtain in a form that is stable over time using conventional emulsifiers. The oil content of the O/W emulsion implementing the emulsifying composition according to the invention is preferably of between 10 and 65% by weight, and preferably of the order of 20 to 55% by weight, relative to the total weight of the emulsion. Vegetable oil or oils of vegetable origin, for example such as sunflower oil and isopropyl palmitate make it possible in particular to obtain stable emulsions, not resulting in creaming or phase separation.

The emulsion implementing the emulsifying composition according to the invention can also comprise a rheology agent in particular as a thickening agent of the aqueous phase, or a gelling agent or a suspending agent, such as for example gums derived from plants like gum arabic, konjac gum, guar gum or their derivatives; gums extracted from algae like alginates or carrageenans; gums derived from microbial fermentation like xanthans, mannans, scleroglucans or their derivatives; cellulose and its derivatives such as carboxymethyl cellulose or hydroxyethyl cellulose; starch and its derivatives such as modified starches, in particular acetylated, carboxymethylated, octenylsuccinate or hydroxypropylated starch; synthetic polymers such as polyacrylic acid or carbomers.

Preferably, the emulsion implementing the emulsifying composition according to the invention comprises a rheology agent selected from the natural polysaccharides from plants or fermentation, optionally modified. Xanthan and its derivatives make it possible in particular to obtain oil-in-water emulsions with very fine droplet sizes, even when used with a content of less than 1% by weight, relative to the total weight of the emulsion.

The emulsions implementing the emulsifying composition according to the invention are, preferably, in the form of a fatty phase dispersed in an aqueous phase, said dispersed fatty phase being in the form of droplets with an average size of less than or equal to 30 μm, preferably of less than or equal to 10 μm.

A small droplet size increases the stability of the emulsion by reducing the flocculation rate of the emulsion, and thus the phase separation rate. The average droplet size depends on a large number of parameters and, as such, is a characteristic that should be controlled and is not intrinsic to the formulation of the emulsifying composition.

The average droplet size can be measured by means of a LEICA DMLS optical microscope at ×10 magnification, followed by a count and calculation of a number average on at least about ten droplets.

The emulsion implementing the emulsifying composition according to the invention can also comprise a preservative selected from benzyl alcohol, dehydroacetic acid and their mixtures.

The emulsion implementing the emulsifying composition according to the invention preferably has a viscosity higher than 3000 mPa·s at 25° C., preferably higher than 5000 mPa·s at 25° C. The viscosity is measured with a Brookfield DV-II+Pro viscometer rotated at a speed of 20 rotations per minute in contact with the product sample. The resistance of the product to this rotational movement is recorded during one minute and converted into “millipascal-second”, usually denoted mPa·s. For each sample, the viscosity is measured three times and the arithmetic mean of the three values is taken.

In order to characterize the sensory properties of the emulsifying compositions according to the invention, sensory descriptors and a corresponding 5-step sensory evaluation protocol are used. These 5 steps correspond to the different phases of application of a treatment product: appearance, handling, application, spreading after 1 minute, and spreading after 2 minutes. During these 5 steps, several sensory descriptors are evaluated by a panel of ten evaluators, in order to compare an emulsion according to the invention with an emulsion according to patent application FR1853362 filed by the present applicant. The emulsions according to the invention differ from the emulsions according to patent FR1853362 in the sensory properties of fluidity, smoothness, spreadability, greasiness, softness, squeakiness, penetrability, and pilling. The emulsions according to the invention provide a less fluid texture, but are still easier to spread and faster to penetrate, as well as having a more greasy feel, but still being smoother, softer, and less squeaky. In addition, there is less pilling, or even no pilling depending on the skins.

Furthermore, the emulsifying composition according to the invention allows the easy production of oil-in-water O/W emulsions that are both very stable and very fine, with buildable textures and a cool, silky, non-greasy feel, even with high levels of dispersed fatty phase. It is thus possible to obtain emulsions having a good emollient effect on the skin as well as a good moisturizing effect on the upper layers of the epidermis.

This emulsifying composition allows in particular the easy production of very fine O/W emulsions with various textures, highly compatible with the skin and additionally having a dry, fresh, silky touch, even with high levels of fatty phase in the emulsion.

Method for Manufacturing a Liquid Emulsion Implementing the Emulsifying Composition

Another object of the present invention consists of a method for manufacturing a liquid oil-in-water emulsion, preferably a Pickering emulsion, in particular for cosmetic use, comprising the following steps:

a) dispersing, in an aqueous phase, an emulsifying composition comprising at least one cyclodextrin in the form of solid particles, said particles having a volume-average size d(4.3) measured by laser granulometry, of between 2 and 20 μm, preferably between 3 and 12 μm, and more preferably between 4 and 8 μm, and at least one emulsifier of natural origin selected from oil-in-water emulsifiers, having a HLB greater than or equal to 8, and more preferably an HLB greater than or equal to 9, in an emulsifier/cyclodextrin ratio of between 0.01:1 and 1:1, preferably of between 0.15:1 and 0.30:1,
b) adding to the mixture obtained in step a) a fatty phase, in an amount of between 10 and 65% by weight, relative to the total weight of the composition, under stirring to allow dispersion of the fatty phase in the aqueous phase in the form of droplets with a number-average size of less than 30 μm, preferably of less than or equal to 10 μm.

According to a variant, the method according to the invention for manufacturing a liquid oil-in-water emulsion, preferably a Pickering-type emulsion, in particular for cosmetic use, comprises the following steps:

a) dispersing, in a fatty phase, an emulsifying composition comprising at least one cyclodextrin in the form of solid particles, said particles having a volume-average size d(4.3) measured by laser granulometry, of between 2 and 20 μm, preferably between 3 and 12 μm, and more preferably between 4 and 8 μm, and at least one emulsifier of natural origin selected from oil-in-water emulsifiers, having a HLB greater than or equal to 8, and more preferably a HLB greater than or equal to 9, in an emulsifier/cyclodextrin ratio of between 0.01:1 and 1:1, preferably of between 0.15:1 and 0.30:1, the fatty phase preferably representing between 10 and 65% by weight, and preferably between 20 and 55% by weight, relative to the final weight of the emulsion,
b) and adding the mixture obtained in step a) to an aqueous phase under stirring in order to allow the dispersion of the fatty phase in the aqueous phase in the form of droplets with an average size of less than 30 μm, preferably of less than or equal to 10 μm.

In the two variants of a method for manufacturing an oil-in-water emulsion, preferably a Pickering emulsion, set out above, it is preferred to use an emulsifying composition according to the invention in which the emulsifier of natural origin is selected from at least one alkyl polyglucoside, at least one mixture of at least one alkyl polyglucoside and at least one fatty alcohol, and at least one non-ethoxylated polyol fatty ester, more preferably selected from the mixtures of at least one alkyl polyglucoside and at least one fatty alcohol.

Use

Lastly, an object of the invention is also the use:
of a cyclodextrin in the form of solid particles having a volume-average size d(4.3) measured by laser granulometry, less than or equal to 20 μm, more preferably between 2 and 20 μm, preferably between 3 and 12 μm, and more preferably between 4 and 8 μm,
and of at least one emulsifier of natural origin selected from oil-in-water-type emulsifiers, having a hydrophilic-lipophilic balance greater than or equal to 8, most preferably greater than or equal to 9, the HLB of the whole emulsifying system of said composition also being greater than or equal to 8, preferably greater than or equal to 9,
   in an emulsion for cosmetic use in order to provide said emulsion with a softer and smoother feel, and to reduce or eliminate pilling.

### EXAMPLES

The invention will be understood better with the help of the non-limiting example of an embodiment described hereinafter.

### Example 1: Emulsion

A “reference” emulsion prepared with an emulsifying composition containing a beta-cyclodextrin marketed under the name “Beauté by Roquette® CD102” by the applicant, the volume-average diameter of which is 94.83 μm, is compared with an emulsion according to the invention, which is obtained using this same beta-cyclodextrin dry-ground beforehand to have a volume-average size d(4.3) of less than 12 μm: the beta-cyclodextrin thus obtained is qualified as “ultra-fine”. The granulometric characteristics of these two beta-cyclodextrins are presented in Table 1.

TABLE 1
granulometric characteristics of the beta-cyclodextrins

| | Beta-cyclodextrin | Beta-cyclodextrin according |
|---|---|---|
| | “Beauté by Roquette ® | to the invention, so-called |
| | CD102” (reference) | “ultra-fine” |
| d(4.3) (μm) | 94.83 | 11.43 |
| d10 (μm) | 21.81 | 2.378 |
| d50 (μm) | 85.81 | 9.716 |
| d90 (μm) | 182.2 | 22.85 |
| CV | 64.7% | 72.3% |

TABLE 2

| Ingredient | Type | reference | invention |
|---|---|---|---|
| Beauté by Roquette | Beta- | 5% | — |
| CD102 | cyclodextrin | | |
| “Ultra-fine” beta- | Beta- | — | 5% |
| cyclodextrin | cyclodextrin | | |
| Sunflower | Oil | 30% | 30% |
| Montanov ™ L | Emulsifier | 1% | 1% |
| Hydroxyethyl cellulose | Thickener | 0.7% | 0.7% |

The two emulsions in Table 2 above are prepared according to the following protocol.

First a gelling agent is dispersed in water under stirring with a deflocculating paddle at 1000 rpm. The water temperature is set at 40° C. when the gelling agent is xanthan gum, and at 70° C. when the gelling agent is hydroxyethyl cellulose.

The beta-cyclodextrin is then wetted in glycerin and the beta-cyclodextrin/glycerin mixture is added to the water/gelling agent mixture under stirring at 1000 rpm, in order to obtain an aqueous phase.

The amount of beta cyclodextrins is set at 5% by weight of the composition. Separately, the MONTANOV L alkyl polyglucoside (INCI: cetearyl alcohol & cetearyl glucose), from the company SEPPIC, is added to sunflower oil or to isopropyl palmitate under magnetic stirring, at 40° C. in order to obtain an oily phase.

The oily phase is then emulsified in the aqueous phase at 40° C. under stirring at 1500 rpm, during 15 minutes.

A preservative (mixture based on benzyl alcohol and dehydroacetic acid) is added.

For each of the emulsions, physical-chemical characteristics are measured, namely viscosity and average droplet size, and sensory characteristics are measured, namely slipperiness, spreadability, greasiness, softness, squeakiness, penetrability, smoothness and pilling.

Viscosity is measured using a Brookfield DV-II+Pro viscometer. A fixed-size moving part (SP2 to SP7 moving parts used according to the viscosity levels following device instructions) is rotated at a speed of 20 rpm in contact with the product sample. The resistance of the product to this rotational movement is recorded during one minute and converted into millipascal-second. For each sample, the viscosity is measured three times and the arithmetic mean of the three values is taken. A spindle adapted to the measured viscosity is selected according to the following ranges: the selected spindle is the SP3 spindle when the viscosity is less than or equal to 5000 mPa·s, SP4 when the viscosity is between 5000 mPa·s and 7000 mPa·s and SP5 when the viscosity is greater than or equal to 7000 mPa·s.

The average size of the droplets is determined by carrying out the arithmetic mean of the droplet sizes measured with an optical microscope at ×10 magnification, on a representative number of droplets, typically at least 10 droplets. The microscope used is a LEICA DMLS.

The sensory characteristics are evaluated by a panel of ten people who are experts in analyzing the texture of cosmetic products.

When spreading the product, two descriptors are evaluated. The product is examined under a lamp, after having placed 50 to 100 μl of the product under examination on the hand, while it is spread for 10 turns.
The smoothness descriptor is evaluated between the 2nd and 5th turn. The fingers slide well over the skin. The product is perceived as a powdery substance on the skin.
Spreading is evaluated by examining the product after having placed 50-100 μl of the product on the hand, while it is spread for 10 turns, under a lamp. Spreading is highest when there is little resistance to movement between the 5th and the 10th turn on the hand.

The last descriptors are evaluated after the 10 turns have been completed. For the following two descriptors, the examination is carried out under a lamp, on the skin, 1 minute after spreading 50 to 100 μl of the product.
The softness descriptor is evaluated by sliding over the skin, a dry and slippery sensation is felt.
The squeakiness descriptor is evaluated by rubbing the thumb with the index finger, resistance is felt, and a squeaky sound is heard.

For the following two descriptors, the examination is carried out under the lamp, on the skin, 2 minutes after spreading 50 to 100 μl of the product.
The penetration descriptor of the product is evaluated by sliding over the skin. A panel of evaluators then evaluates the amount of product residue recovered.
The pilling is evaluated by carrying out a mechanical rubbing action on the skin, the product leads to the formation of pills.

The two emulsions have substantially equal viscosities, around 12,000 mPa·s, and have droplet sizes of less than 10 μm.

Table 3 presents the sensory perceptions obtained with the emulsion prepared with the emulsifying composition according to the invention, compared with the sensory perceptions obtained with an emulsion prepared with a reference emulsifying composition.

The criteria for improved sensory properties in the emulsion according to the invention are: smoothness, spreading, greasy, soft, squeaky, penetrating, pilling.

TABLE 3
improved sensory perceptions with
respect to the reference emulsion

| | Emulsion according to the invention, |
|---|---|
| Sensory | with ultra-fine beta-cyclodextrin, |
| perception | compared with the reference emulsion |
| Smooth | Smoother |
| Spreading | Easier |
| Softness | Softer feel |
| Squeaky | Notably less squeaky |
| Penetrating | Faster penetration |
| Pilling | No pilling or less pilling |

1. An emulsifying composition, preferably for cosmetic use, capable of obtaining an oil-in-water-type emulsion, comprising:
a) at least one cyclodextrin in the form of solid particles, said particles having a volume-average size d(4.3) measured by laser granulometry, of between 2 and 20 μm, preferably between 3 and 12 μm, and more preferably between 4 and 8 μm,
b) and at least one emulsifier of natural origin selected from oil-in-water-type emulsifiers, having a hydrophilic-lipophilic balance greater than or equal to 8, most preferably greater than or equal to 9.

2. The emulsifying composition according to claim 1, wherein the cyclodextrin particles have a volume-size distribution, measured by laser diffraction granulometry, the characteristic diameters d(10), d(50) and d(90) of which are such that:
a) the diameter d(10) is of less than or equal to 5.0 μm, preferably of less than or equal to 2.5 μm, and/or
b) the diameter d(50) is of less than or equal to 15.0 μm, preferably of less than or equal to 10.0 μm, and/or
c) the diameter d(90) is of less than or equal to 30.0 μm, preferably of less than or equal to 25.0 μm.

3. The emulsifying composition according to claim 1, wherein the solid cyclodextrin particles have a volume-size distribution, measured by laser diffraction granulometry, having a coefficient of variation of less than or equal to 100%, preferably of less than or equal to 90%, and most preferably of less than or equal to 73%.

4. The emulsifying composition according to claim 1, wherein said at least one emulsifier of natural origin has a hydrophilic-lipophilic balance (HLB) between 8 and 20, preferably between 9 and 16, and more preferably between 11 and 14.

5. The emulsifying composition according to claim 1, wherein the average HLB of the overall emulsifying system of said composition is greater than or equal to 8, preferably greater than or equal to 9.

6. The emulsifying composition according to claim 1, wherein the emulsifier of natural origin is present in the emulsifying composition in an emulsifier/cyclodextrin ratio of between 0.01:1 and 1:1, preferably of between 0.05:1 and 0.5:1, more preferably of between 0.10:1 and 0.35:1 and even more preferably of between 0.15:1 and 0.30:1.

7. The emulsifying composition according to claim 1, wherein said cyclodextrin is selected from alpha-, beta- and gamma-cyclodextrins, preferably it is a native beta-cyclodextrin.

8. The emulsifying composition according to claim 1, wherein the emulsifier of natural origin is an oil-in-water emulsifier selected from: the alkyl polyglucosides; the mixtures of at least one alkyl polyglucoside and at least one fatty alcohol; the non-ethoxylated polyol fatty esters, in particular from the glycerol, polyglycerol, sorbitol, sorbitan, anhydrohexitol fatty esters, preferably from isosorbide, mannitol, xylitol, erythritol, maltitol, sucrose, glucose, polydextrose fatty esters, fatty esters of hydrogenated glucose syrups, dextrin fatty esters and fatty esters of hydrolyzed starches.

9. The emulsifying composition according to claim 1, wherein the emulsifier of natural origin is an oil-in-water emulsifier selected from the C12-C20 alkyl polyglucosides, with the following general formula (I): R1-O—(R2-O)p-(S)n, wherein:
R1 designates a linear or branched alkyl and/or alkenyl radical having about 12 to 20 carbon atoms
R2 designates an alkylene radical comprising 2 to 4 carbon atoms,
p takes a value ranging from 0 to 3, and preferably equal to zero,
S denotes glucose, fructose or galactose, and more preferably glucose,
n denotes a value ranging from 1 to 15

10. The emulsifying composition according to claim 8, wherein the O/W emulsifier of natural origin is a mixture consisting of at least one alkyl polyglucoside and at least one fatty alcohol, preferably at least one C12-C20 alkyl polyglucoside and at least one linear or branched fatty alcohol having a total number of carbon atoms ranging from 8 to 24, and most preferably a mixture of C14-C22 fatty alcohol and C12-C20 alkyl glucoside.

11. The emulsifying composition according to claim 1, comprising:
a) 40% to 95% of at least one cyclodextrin in the form of solid particles, said particles having a volume-average size d(4.3) measured by laser granulometry, of between 2 and 20 μm, preferably between 3 and 12 μm, and more preferably between 4 and 8 μm,
b) 5% to 40% of at least one emulsifier of natural origin selected from oil-in-water-type emulsifiers, having a hydrophilic-lipophilic balance greater than or equal to 8, most preferably greater than or equal to 9,
c) And 0% to 40% of at least one polyol.

12. A liquid emulsion, preferably an oil-in-water Pickering emulsion, wherein it contains at least one cyclodextrin in the form of solid particles, said particles having a volume-average size d(4.3) of between 2 and 20 μm, preferably between 3 and 12 μm, and more preferably between 4 and 8 μm, and at least one emulsifier of natural origin selected from oil-in-water-type emulsifiers, having a hydrophilic-lipophilic balance greater than or equal to 8, most preferably greater than or equal to 9, in an emulsifier/cyclodextrin ratio of between 0.01:1 and 1:1, preferably between 0.15:1 and 0.30:1.

13. The emulsion according to claim 12, wherein it is presented in the form of a fatty phase dispersed in an aqueous phase, said dispersed fatty phase being provided in the form of droplets with an average size of less than 30 μm, preferably of less than or equal to 10 μm.

14. The composition according to claim 12, having a viscosity higher than 3000 mPa·s at 25° C., preferably higher than 5000 mPa·s at 25° C.

15. A method for manufacturing a liquid emulsion, preferably an oil-in-water Pickering emulsion, comprising the following steps:
a) dispersing, in an aqueous phase, an emulsifying composition comprising at least one cyclodextrin in the form of solid particles, said particles having a volume-average size d(4.3) measured by laser granulometry, of between 2 and 20 μm, preferably between 3 and 12 μm, and more preferably between 4 and 8 μm,
and at least one emulsifier of natural origin selected from oil-in-water-type emulsifiers, preferably having a hydrophilic-lipophilic balance greater than or equal to 8, most preferably greater than or equal to 9, in an emulsifier/cyclodextrin ratio of between 0.01:1 and 1:1, preferably of between 0.15:1 and 0.30:1,
b) adding a fatty phase to the mixture obtained in step a), in an amount of between 10 and 65% by weight, relative to the total weight of the composition, under stirring to allow dispersion of the fatty phase in the aqueous phase in the form of droplets with a number-average size of less than 30 μm, preferably less than or equal to 10 μm.

16. A combined use of a cyclodextrin in the form of solid particles having a volume-average size d(4.3) measured by laser granulometry, of between 2 and 20 μm, preferably between 3 and 12 μm, and more preferably between 4 and 8 μm,
and of at least one emulsifier of natural origin selected from oil-in-water-type emulsifiers, having a hydrophilic-lipophilic balance greater than or equal to 8, most preferably greater than or equal to 9, in an emulsion for cosmetic use in order to provide said emulsion with a softer and smoother feel, and to reduce or eliminate pilling.
